(19) United States
(12) Patent Application Publication
(10) Pub. No.: US 2021/0346148 A1
(43) Pub. Date: Nov. 11, 2021

(54) SUTURE CONSTRUCT AND METHOD OF TISSUE FIXATION

(71) Applicants: Smith & Nephew, Inc., Memphis, TN (US); Smith & Nephew Orthopedics AG, Zug (CH); Smith & Nephew Asia Pacific Pte. Limited, Singapore (SG)

(72) Inventors: Ali Hosseini, Quincy, MA (US); Christopher David MacCready, Medfield, MA (US); Paul McGovern, Hanson, MA (US); Chun Liu, Brookline, MA (US); Geoffrey Ian Karasic, Milton, MA (US)

(21) Appl. No.: 17/308,228

(22) Filed: May 5, 2021

Related U.S. Application Data
(60) Provisional application No. 63/020,718, filed on May 6, 2020.

(51) Int. Cl. A61F 2/08

(52) U.S. Cl. CPC A61F 2/0811; A61F 2002/0829; A61F 2002/0876; A61F 2250/0012; A61F 2002/0852

(57) ABSTRACT

An adjustable fixation system for coupling a tissue or graft to bone is disclosed including a suture construct including a first limb, a second limb and a body therebetween. The body is continuously braided with the first and second limbs and has a longitudinal passage therethrough. The first and second limbs define a braided portion including a braided core. The suture construct is configured to form at least one adjustable loop formed by passing the first limb between braids of the suture construct, along the longitudinal passage and back out between braids of the adjustable suture construct. Tension on at least one of the first or second limbs reduces a diameter of the longitudinal passage around a length of the first limb and thereby the braided core disposed along the body longitudinal passage and knotlessly locks the adjustable loop.

### CROSS-REFERENCE TO RELATED APPLICATIONS

This application claims benefit to Provisional Patent Application No. 63/020,718; filed May 6, 2020, titled “SUTURE CONSTRUCT AND METHOD OF TISSUE FIXATION” and incorporated by reference in its entirety.

### FIELD

The present disclosure relates to methods and devices for tissue fixation in a surgical repair.

### BACKGROUND

Soft tissue (such as ligaments and tendons) that has ruptured or torn may be repaired or replaced surgically. In some tissue repairs, an adjustable loop construct may be coupled to the tissue, the loop formed by threading the suture through itself along a longitudinal passage of the suture. Tensioning of the suture may cinch the longitudinal passage and knotlessly lock the suture loop so that it is no longer adjustable. This is similar to a Chinese finger trap. Sutures that form these suture loops are coreless along their entire length. During cyclic loading, typical during daily use of the joint or tissue, these coreless sutures may slowly elongate, and/or the locking passage may relax over time and the suture loop may extend, loosening the tissue repair. There is therefore a need for an adjustable loop construct that resists elongation or loosening over time.

### SUMMARY

Described herein are various improvements in methods and devices for tissue fixation using an adjustable fixation loop that may be knotlessly locked. The adjustable fixation loop includes a braided suture that includes a braided core (a core that includes braids therein) that is briefly interrupted by a locking length that is defined by a longitudinal passage or void therealong. This adjustable fixation loop forms a knotless locking loop that provides improved elongation resistance during physiological loading. Adjustable fixation loop may form a portion of a graft suspension system or tissue repair system that may include an anchor. These and other features and advantages will be apparent from a reading of the following detailed description and a review of the associated drawings. It is to be understood that both the foregoing general description and the following detailed description are explanatory only and are not restrictive of aspects as claimed.

More specifically an example embodiment disclosed herein may include a tissue repair system including a suture construct with a first and second braided core segment and a locking length therebetween. The locking length is continuously braided with the first and second braided core segment, the locking length defining a locking passage therealong. The locking length is preferably short relative to the remaining suture construct. The suture construct forms at least one adjustable loop, formed by placing the first braided core segment into and along the locking length. Tension on at least one of the first or second braided core segments knotlessly locks the locking length around the first braided core segment disposed therein.

In some example embodiments, the suture construct includes at least two adjustable loops with the first and second braided core segment disposed within the locking length. In this example, tension on at least one of the first or second braided core segments knotlessly locks the locking length around both the first and second braided core segment disposed therein. In this example embodiment, the first and second braided core segments pass through the locking length in opposite directions.

In some example embodiments, the adjustable loop may be operatively coupled to an anchor. In some example embodiments, the adjustable fixation loop may provide a suspension means for suspending a ligament in a bone hole. In some example embodiments, the locking passage may define a cradle for operatively coupling with a ligament graft. In some example embodiments, the locking length has a first length and the first and second braided core segments in combination define a second length, the second length more than twice as long as the first length. In some other embodiments, the suture construct is trimmed after knotlessly locking the construct and in this trimmed configuration; the second length is more than three times as long as the first length.

In some example embodiments, all of the strands in the first and second braided core segment are disposed along the locking length also. In some example embodiments, the adjustable fixation construct may include a second adjustable loop formed by passing the second limb between braids of the adjustable suture construct, along the locking length of the body and back out between braids of the adjustable suture construct such that the locking passage includes a length of the first and second limbs including, by definition, the braided core of both the first and second core. In some example embodiments, the first and second limbs pass through the locking length in opposite directions.

Another example embodiment disclosed herein includes a knotless tissue fixation system including a tissue anchor and a continuously braided suture operatively coupled to the tissue anchor, the braided suture having a braided core therealong, interrupted by a short coreless portion, defining a locking length. The locking length defines a predetermined limited length sufficient to both knotlessly and securely lock around a length of suture disposed within the locking length upon tension being applied to the continuously braided suture.

In some example embodiments, the braided suture including the braided core may be disposed along the locking length to form a knotless locking suture loop. In some example embodiments, the suture disposed along the locking length is a separate suture, independently formed from the braided suture. In some example embodiments, the tissue anchor may be an all-suture anchor that is deformable to selectively couple with a bone hole. In some example embodiments, the tissue anchor may be a rigid anchor. In some example embodiments, the braided suture has the same number of strands in the braided core segment as the locking length. In some example embodiments, the suture enters and exits the coreless portion and thereby the locking length between braids of the braided suture at opposing ends of the locking length. In some example embodiments, the locking length has a length that is limited to a minimum length sufficient to securely knotlessly lock around the suture. In some example embodiments, the locking length has a length that is substantially shorter than a length of suture with a braided core and tension on the suture preferentially elongates the locking length. In some example embodiments, the locking length is limited to a minimum length for locking around the suture while the braided core portion is maximized to minimize elongation or strain over time. The locking length may have a first length sufficient to lock around the suture disposed therein, and the entire continuously braided suture defines a second length, the second length being more than twice the first length.

An example method of coupling a first tissue to a second tissue is also disclosed herein, including coupling a suture to the first tissue, and coupling the suture to a second tissue, wherein the suture includes a locking length extending between first and second braided core segments of the suture. Both braided core segments and the locking length are continuously braided with each other and have equal number of strands to each other therealong. One of the braided core segments are placed into and along the locking length. A first tension is then applied to tension a first limb of the suture to draw the first tissue towards a target location; and a second tension is then applied to the suture, to knotlessly lock the locking length around the braided core segment disposed therein and thereby knotlessly fix the first tissue to the second tissue.

In some example methods, coupling the suture to the second tissue may include engaging an anchor with a second tissue, the anchor operatively coupled to the suture. In some example methods, another braided core segment of the suture is placed into and along the locking length before applying a second tension. In some example methods, another braided core segment of the suture may be placed into and along another locking length of the suture before applying a second tension.

An example method of knotlessly fixing a tissue or graft is also disclosed including: coupling a suture to the tissue or graft, wherein the suture includes a locking length extending between a first and second limb of the suture; wherein both limbs and the locking length are continuously braided with each other so as to have a uniform number of braids therealong; and wherein both limbs include braids disposed within and around a core of both limbs and wherein the locking length defines a longitudinal passage, and therefore absent braids along its core. The method also includes applying a first tension to the first limb to draw the tissue or graft towards a target location. The method also includes applying a second tension to the suture with the first limb disposed within the longitudinal passage, so as to contract the longitudinal passage around the first limb and knotlessly lock the loop and thereby the tissue or graft. The example method may also include coupling a suture to a tissue or graft by suspending a graft over the locking length. The example method may also include coupling a suture to a tissue or graft by threading a length of the suture through the tissue or graft. The example method may also include inserting an anchor into a tissue associated with the target location, the anchor operatively coupled to the suture and deploying the anchor to fix the anchor with the tissue associated with the target location such that applying the second tension knotlessly locks the tissue or graft with the anchor and thereby the target location. The anchor may be selected from a group consisting of an all-suture anchor; a rigid anchor and a button anchor. Deploying the anchor may be performed before applying the second tension. Deploying the anchor may be performed after applying the second tension.

### BRIEF DESCRIPTION OF THE DRAWINGS

The disclosure will be more fully understood by reference to the detailed description, in conjunction with the following figures, wherein:

FIG. 1A illustrates a suture construct with a braided core having a coreless portion along the suture construct length, in accordance with this disclosure;

FIG. 1B illustrates a cross section of the braided core portion of the suture construct, in accordance with this disclosure;

FIG. 1C illustrates a cross section of the coreless portion of the suture construct, in accordance with this disclosure;

FIG. 2A illustrates an example adjustable loop formed from a suture construct having a braided core with a discrete coreless portion, in accordance with this disclosure;

FIG. 2B illustrates an example adjustable loop formed from a suture construct having a braided core with two discrete coreless portions, in accordance with this disclosure;

FIG. 2C illustrates an example adjustable suture construct having a braided core with at least one discrete coreless portions, in accordance with this disclosure;

FIG. 3A illustrates an example tissue repair construct with an adjustable suture loop formed from a suture construct having a braided core, interrupted with a coreless portion, in accordance with this disclosure;

FIG. 3B illustrates a cross section of the locking length shown in FIG. 3A;

FIG. 4A illustrates an example tissue repair construct embodiment with an example adjustable loop formed from a suture construct having a braided core with a two discrete coreless portions, in accordance with this disclosure;

FIG. 4B illustrates an example tissue repair construct schematic with two tissue anchors with an adjustable loop formed from a suture construct having a braided core with a two discrete coreless portions, in accordance with this disclosure;

FIG. 4C illustrates a cross section of the suture construct shown in FIG. 4A or 4B;

FIG. 5A and 5B illustrates an example soft anchor construct with an adjustable loop formed from a suture construct having a braided core portion with a discrete coreless portion therealong, in accordance with this disclosure; and

FIG. 6 illustrates an example rigid anchor construct with an adjustable loop formed from a suture construct having a braided core interrupted by a coreless portion, in accordance with this disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open-ended, include the listed parts, and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

The term “locking length” defines a segment of a continuous length of a braided suture that includes a longitudinal passage (open space or void) along the suture. The locking length is defined in that both ends of the longitudinal passage terminate with a transition to a different segment of the continuous length of braided suture that includes braids disposed along the longitudinal passage in place of at least some of the opening space or void that defines the longitudinal passage. Therefore, this different or second segment does not include a longitudinal passage; or the longitudinal passage has become significantly smaller. This second segment may be called a “braided core” segment, as the core may include braids where they are absent in the locking length. The entire continuous length of the braided suture may be a length between 5-200 inches; however, locking length is preferably limited in length and may include a length up to a maximum of about 2 inches depending on the procedure and construct. Preferable ranges for many procedures and constructs may be between ¼ to 2 inches. Locking length is sufficiently long to provide a knotless lock around a length of suture disposed along the locking length upon tension being applied to the locking length.

In general, the entire length of braided suture that includes both at least one locking length and at least one braided core segment may be provided with an “initial length” and may be trimmed and altered during the procedure to a “final length”. For example, the length of suture may be supplied with an “initial length” that may include suture limbs that may be used to manipulate the length of suture. For example, these suture limbs may be used to apply tension to the lengths of suture and knotlessly lock the construct. During the procedure, the limbs may be trimmed and splices or knots may be formed. As such, the length of suture may be shortened to a “final length”, shorter than the initial length.

To exhibit enhanced resistance to elongation or strain over time, it is preferable that the total length of second segments (braided core segments) in the “final length” as defined above, be maximized. Relative to a suture with a hollow core along most or all of its length, a length of suture with a braided core exhibits a high resistance to elongation during cyclic loading post-surgery. It follows therefore that the length of the locking length should be minimized to maximize the braided core length, while the length of the locking length must be sufficiently long, to knotlessly lock the loop or repair construct. It is preferable that the total length of second segments be at least as long as the locking lengths. As way of an example, if a “final length” configuration includes a single “locking length” that is 1.5 inches, then the remaining length of suture in the “final length” construct including only a braided core length, is preferably at least 1.5 inches long. In some embodiments this remaining length may be significantly more than 1.5 inches, such as for example double loop constructs (discussed later herein), the remaining length may be at least 3 inches. Otherwise stated a ratio of locking length to braided core length is greater than 1:1, and more preferably greater than 1:2 and may be up to 1:100 depending on the application.

The term “knotlessly lock” defines a configuration of the suture construct with a locking length that prevents suture present within and along the longitudinal passage of the locking length from sliding along the locking length. The locking length, under tension is configured to elongate and narrow and forms a Chinese finger-trap style lock around a suture present within the locking length. This does not preclude a knot at other locations along the suture construct. For example, a knot may couple the suture to an anchor or couple the suture to a biological tissue and still meet the definition. Suture constructs that use Chinese finger-trap style knotless locking constructs typically include suture that have hollow cores along their entire length.

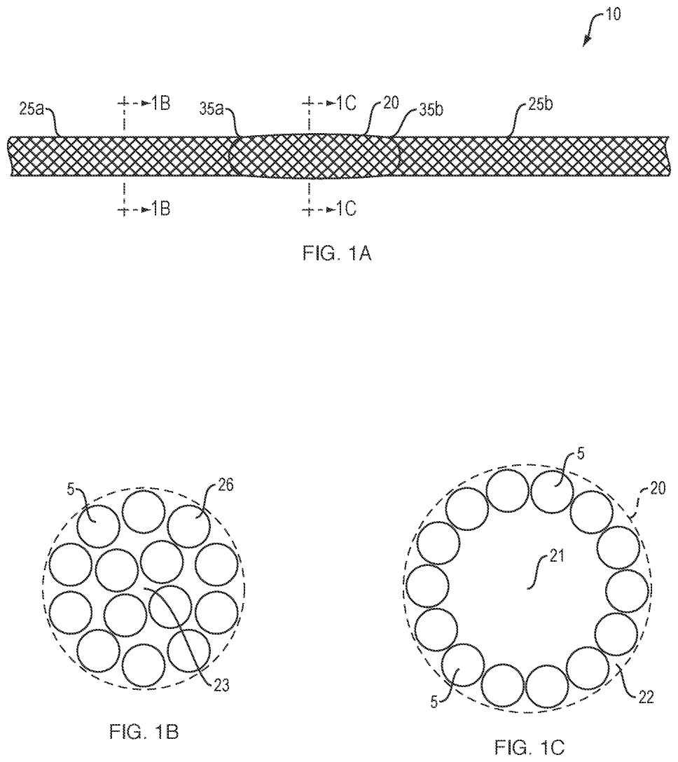

Referring now to FIG. 1A, a portion of a braided suture 10 is illustrated, and includes a locking length 20 disposed between two braided core lengths 25a and 25b. The braided suture 10 is continuously braided between the locking length 20 and braided core lengths 25a, 25b. The suture 10 is preferably formed with the same number of strands 5 between the locking length 20 and braided core lengths 25a, 25b. Each individual strand 5 may not change in the type and thickness as it extends along and between the locking length 20 and braided core lengths 25a, 25b, (beyond normal manufacturing tolerances). This does not preclude each individual strand from being different from other strands in material or thickness for example. A strand 5 preferably does not alter in material or thickness along its length, particularly as it relates to the transitions between the locking length 20 and braided core lengths 25a, 25b.

Referring to both FIG. 1A and 1C, locking length 20 includes an outer wall 22 formed by strands 5, defining the boundary of a core 21, the core 21 absent braids therealong. Outer wall 22 may define a tubular shape. Outer wall 22 is formed of a plurality of strands 5, braided around each other forming a continuous braided outer wall of the braided suture 10 along locking length 20. As shown outer wall 22 includes a single layer or ring of strands 5. Alternative configurations (not shown) may include a plurality of strand layers or concentric rings that may be intertwined with each other. Referring to both FIG. 1A and 1B, braided core lengths 25a and 25b include an outermost wall section 26 and a braided core 23 that includes strands, 5. Outermost wall section 26 may include strands 5 that define a tubular shape. FIG. 1B is a simplified representation of a single instantaneous cross section of this segment 25a, and in reality there may be no distinct tubular shape formed that is separate from the strands in the core 23, as such; all of the strands 5 shown in FIG. 1B weave around each other in a pattern, such that the strands 5 continuously move between the outermost section 26 and core section 23 along the braided suture length. Outermost wall section 26 essentially represents an instantaneous outer-most layer of the segments 25a and 25b.

While represented as a circular cross section, other cross section configurations may be formed, such as an oblong cross section, having a width larger than height. Different cross sections may be formed during braiding, or during a secondary manufacturing process that flattens or re-shapes the suture cross sections. As shown, the cross sections are shown in a simplified form. In reality, suture and the flexible strands that form the suture may naturally deform and relax to form a different cross section of a different shape (non-circular configuration) which is not precluded from this disclosure. Stated alternatively, this three-dimensional structure preferably defines a cross section shape and includes a predefined inner space or resident volume along the locking length 20. The resident volume is a volume that may be formed by the three-dimensional structure in its manufactured state, but when the sides of the structure are compressed or relaxed, the resident volume may become smaller or disappear altogether. The cross section may also become non-circular. The term and shapes shown herein do not preclude the structure folding or collapsing in on itself, to temporarily obscure the presence of the resident volume and temporarily alter the cross section shape. The relaxed configuration of a shape as manufactured is not considered different from this disclosure. In addition, the locking length may deform (be dilated) in order to receive a length of suture therethrough, and knotlessly lock the construct. This deformation also does not preclude a cross section shape.

Locking length 20 transitions of braided core segment 25a, 25b at transitions 35a and 35b. There is no substantial interruption in the strands 5 as braided, at transitions 35a and 35b. Preferably, the transition is free of any splicing, gluing, or other fastening between segments 25a, 25b and locking length 20. Preferably, the braiding is continuous between all the segments along the suture 10 and the number of strands 5 is uniform such that the outer wall section 26 includes less strands 5 than tubular section 22, as some of the strands 5 from outer wall 22 are braided to move centrally and form the braided core 23 along the braided core segment 25a and 25b. A maximum outer diameter of the locking length 20 may be larger than a corresponding outer diameter of the braided core segment 25a and 25b. This may be due to the radial shifting of strands 5 in outer wall section 22 relative to outer wall section 26. In addition, the braiding pattern or tension on the braiding machine may be altered to weave the strands 5 more loosely, (lower pick count) to form a reduced strand count per length of the suture. This may aid in readily forming entrance and exit points for a suture at or adjacent to transition points 35a and 35b, discussed in more detail in another portion of this application. The core 21 may also preferably be increased with this lower pick count for at least one length suture to be placed along the core 21, also discussed in more detail later. Strands 5 are preferably consistent in material and dimensions along suture.

Returning to FIGS. 1A, 1B and 1C, and as an example, the braided suture 10 may be formed of fourteen strands integrated in a pattern as described herein. This is exemplary and up to sixty-four strands are considered. Along braided core segment 25a, at any instant cross section as represented in FIG. 1B, ten strands may form the outermost wall section 26 while four strands extend along the core 23. As described earlier, these strands 5 are not limited to the tubular section 26 and core portion 23 along the length of braided core segment 25a. In practice, these strands 5 move around each other according to a braiding pattern and location of braiding spools, and weave in and out of the core portion 23 and outermost wall section 26 continuously. At transition point 35a the braiding pattern changes to move all strands 5 to or towards the outer wall section 22 to form locking length 20 with a longitudinal passage 21 therealong. All fourteen strands 5, continuing with the example, are now braided round the core 21 that preferable free of strands 5. The fourteen strands 5 continue to weave around each other to form a braided outer wall section 22 defining the longitudinal or locking passage. At transition point 35b, the braiding pattern may return to the braiding pattern as described for braided core segment 25a.

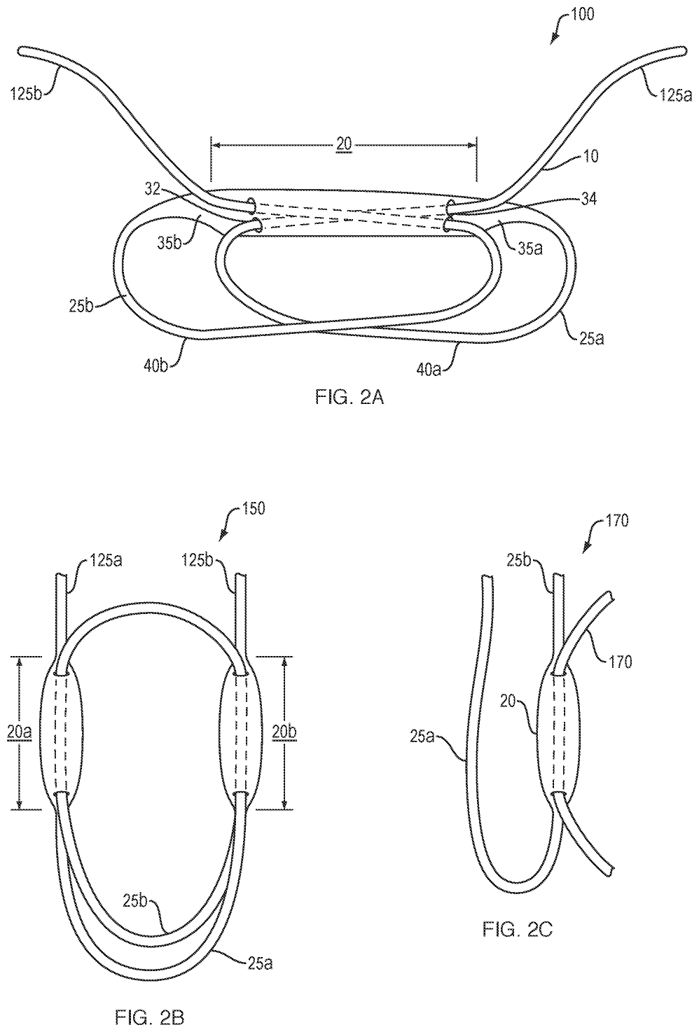

FIG. 2A represents an example adjustable suture loop construct 100 formed with suture 10, according to the present teachings. As shown two loops, 40a and 40b are shown. In alternative embodiments, a single loop may suffice. Shown is a length of suture such as braided suture 10 having a first braided core segment 25a and a second braided core segment 25b. First and second braided core segments 25a, 25b extend up to and include first and second limbs 125a, 125b. The suture 10 is braided and has a locking length 20 limited as described herein. First and second braided core segments 25a and 25b include braided cores 23 as described herein. First and second apertures 32 and 34 are defined in the braided suture 10 at first and second locations that are preferably adjacent transition areas 35a and 35b. First and second apertures 32 and 34 may be formed when constructing the loops 40a and 40b, by inserting a limb between strands of the braided wall. Apertures 32 and 34 may also be partially formed during the braiding process as looser portions between pairs of strands 5 of the suture 10.

A first limb end 125a of the suture 10 may be passed through the first aperture 32 and through longitudinal passage (core 21) of locking length 20 and out the second aperture 34. The second limb end 125b may be passed through the second aperture 34, through and along the locking length core 21 and out the first aperture 32. In alternative embodiments, the loops may be formed at ends of the locking length, such that first limb end 125a of the suture 10 may be passed through the second aperture 34 and through core 21 of locking length 20 and out the first aperture 32. The second limb end 125b may be passed through the first aperture 32, through and along the locking length core 21 and out the second aperture 34. In further alternative embodiments, the second limb 125b may have its own discrete entrance apertures and exit apertures (as shown) slightly spaced away from apertures 32 and 34. As shown in FIG. 2A, each loop is formed with the limbs 125a and 125b crossing over each other in opposite directions through core 21. In further alternative embodiments, limbs 125a and 125b may form loops by entering the same side of the locking length 20, through entrance 32 for example, and extending along core 21 in the same direction.

Adjustable suture construct 100 may be coupled to a biological tissue and tension on at least one of the limbs 125a or 125b may reduce the corresponding loop length and draw the biological tissue in apposition with a target area or tissue. For example, limb 125a may be coupled to a first and a second side of a tear through tissue before forming the loop construct, such as loop construct 100, and thereby before threading limb 125a through and along the locking length 20. Once at least one adjustable loop is formed, similar to construct 100, tension on limb 125a may draw the first and second side of a tear together. Further tension on limb 125a, may reduce a diameter of the core 21 of the locking length 20 around the suture limbs disposed therealong and knotlessly lock the two tissue sides together. This example may be duplicated with both limbs 125a, 125b coupling to tissue, and thereby using a double loop. The locking length 20 is configured to cinch around a suture disposed along the locking length core. Similar to a Chinese finger trap, the locking length 20 secures or cinches around the suture disposed along the locking length core and prevents this suture from sliding along the locking length core. The locking length 20 preferably cinches around a length of the braided core 25a, 25b, and not a length of the suture 10 that is includes a longitudinal passage such as a locking length 20. The locking length may have a first length, and the entire adjustable suture construct 100 in a final length configuration, as defined herein may have a second length including the first length. The ratio of the first length to the second length is less than 1:1. The ratio of the first length to the second length is preferable less than 1:2 and may be up to 1:100 depending on the application. The first length is preferable significantly less than the second length.

FIG. 2B illustrates another example of an adjustable suture loop 150 with two discrete locking lengths 20a, 20b along the loop 150. FIG. 2C illustrates another example of an adjustable suture loop 170 with at least one locking length along the loop 170. In this example a separate suture or flexible strand, 175 that may be coupled to a tissue (not shown) and may extend along the locking length 20.

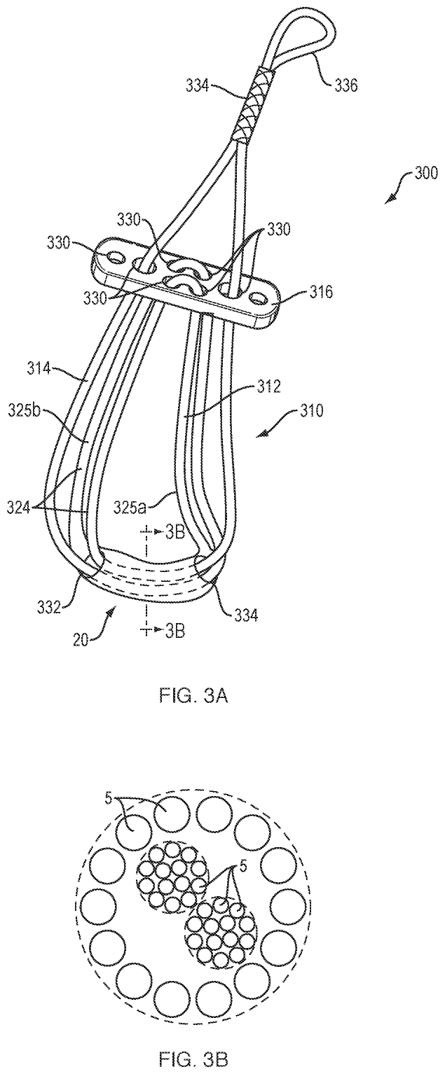

FIG. 3A illustrates an example graft suspension construct 300 including an adjustable suture loop construct 310 formed from suture construct 10. Suture loop construct 310 may be similar to the loop construct 100, with the addition of cortical button 316, which is a form of tissue anchor. Cortical buttons 316 have a first configuration for inserting the button 316 into and along a bone-tunnel and are then deployed or flipped to engage a cortical surface of the bone. In this configuration, locking length 20 may form a cradle, and a graft may be wrapped over the locking length 20. Construct 300 is described in detail in commonly owned U.S. Pat. No. 10,383,617, herein incorporated by reference, in its entirety. The graft suspension construct 300 comprises a suture construct 310 having a first loop 312 and a second loop 314 (together, the suspension loop 324) suspended from anchor 316. The suture construct 310 includes a locking length 20 with two limbs 325a and 325b extending therefrom. A length of the suture 310 may be about 46 inches. The cortical button 316 may be of conventional construction for securement to the outside of a bone, such as a cortical button. The graft suspension device 300 locking length 20 defines a cradle and may be located substantially midway along the length of the suture 310.

Cortical button 316 may include a plurality of apertures 330, which may be eight apertures, extending therethrough and configured for the passage of the suture 310 therethrough. More specifically the cortical button 316 receives the limbs 325a and 325b therethrough. In this embodiment, cortical button 316 does not receive the locking length 20 therethrough. Similar to construct shown in FIG. 2A, limbs 325a and 325b of the suture 310 are braided core segments and are threaded through the locking length 20 to complete the suspension loop 324. Limbs 325a and 325b may be joined together to form a single tail 334, described in detail in commonly owned U.S. Pat. No. 10,383,617, herein incorporated by reference, in its entirety. Single tail 334 may therefore also include a locking length 20. Routing the suture construct 310 through the plurality of apertures 330 as shown keeps the various strands of suture limbs 325a and 325b separated, making them less likely to bunch or tangle. Other cortical buttons known in the art may include fewer apertures therethough, and a plurality of lengths of suture may extend through the same apertures, while not departing from the invention. The single tail 334 may be further adapted to form a finger loop 336 to provide a means whereby the surgeon can adjust the distance of the locking length 20 from the anchor 316, and/or shortening the length of the suspension loop 324, either before or during the surgical operation. Advantageously, the finger loop 336 may be used with just one hand.

Loops 312 and 314 are formed by passing limbs 325a and 325b through gaps between adjacent strands of the suture 310. A length of the locking length 20 is selected to be sufficiently long enough to accommodate a ligament fixation graft suspended thereover, but short enough to facilitate efficient adjustment of the suspension loop 324 while in a bone tunnel. As described herein, having a shorter locking length 20 allows for a maximum length of the loop construct 310 to include braids along the core. Locking length 20 may be sufficiently long to cradle the graft (not shown). The graft suspension device may be configured to fix an ACL ligament graft within the knee and as such is expected to undergo considerable physiological cyclic loading as the patient articulates his or her knee. In alternative embodiments, adjustable construct 100 or 310 may include a second anchor, operatively coupled to the constructs. This construct may be used to repair a knee meniscus tear, or stabilize a shoulder joint, or tibia and fibia bones close to the ankle syndesmosis. Having a maximum length of braided core is preferential to resist strain or elongation during this physiological loading. Suture elongate may reduce tension on the graft and tissue or graft stabilization over time.

FIG. 3B is a representation of a cross section through locking length 20 of FIG. 3A. The strands are not shown to scale, for simplicity. The strands 5 are all preferably the same size between the outer rim 22 and the two braided core portions extending therethrough.

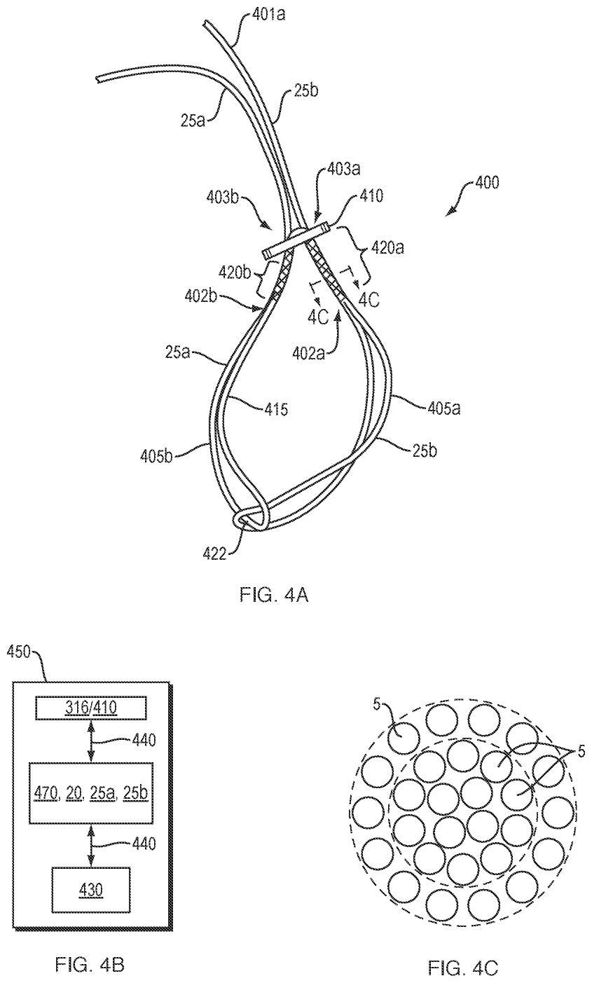

FIG. 4A shows an alternative configuration of an adjustable suture loop construct 415 operatively coupled to a cortical button 410 that may be used to fix a graft for ACL reconstruction procedures or other tissue of graft stabilization. This embodiment includes two locking lengths 420a and 420b, along the construct 415, which may be disposed through or directly adjacent cortical button 410. The cortical button 410 or anchor may provide cortical bone fixation of the graft or tissue and may be similar to other cortical buttons known in the art. Cortical buttons have a first configuration for inserting the button into and along a bone tunnel and are then deployed or flipped to engage a cortical surface of the bone. Similar to previously described embodiments, adjustable loop construct 415 has an adjustable length that is configured to form a knotless locking loop. Construct 415 includes two discrete locking lengths 420a and 420b along the adjustable loop 415 that are separated by a braided core length. Pulling on the free limbs 401a and 401b, reduces the loop length first and then upon further tension each locking length 420a and 420b may constrict, and knotlessly lock the adjustable loop construct 415. Free limbs 401a, 401b may then be trimmed, to form a final length, as defined herein. Locking length 420a, 420b locks around a braided core length. Locking length is sufficiently long to knotlessly lock the adjustable construct 415, but is still substantially shorter than the final length.

In this example embodiment, each limb 401a and 401b forms an eyesplice loop, loop 405a and 405b, that is linked 422 during construct assembly. Stated in another way limb 401a extends from locking length 420a at point 402a, forms a loop 405a and then enters locking length 420a at point 402a and exits at point 403a. Similarly, limb 401b extends from locking length 420b and at point 402b, forms a loop 405b and then enters locking length 420b at point 402b and exits at point 403b. Points 402a 402b, 403a and 403b define ends of the locking lengths or suture transition points, similar to transition points 35a and 35b as described in FIG.1. In some embodiments, link 422 may operatively couple to a second anchor.

Represented in FIG. 4B is a tissue repair assembly 450 including an adjustable suture construct 470 including a locking length 20 and braided core length 25a, 25b. Suture construct 470 is operatively coupled 440 to both a first anchor such as cortical anchor 316, 410 and also operatively coupled 440 to a second anchor 430. The two anchors may be coupled to opposite ends of the construct 470. Operatively coupling may include threading the suture through the anchors 316, 410, 430, or tying knots for example. Second anchor 430 may be a cortical anchor that is the same or different from anchor 316, 410. Second anchor 430 may be a soft anchor, formed of a soft flexible material such as braided suture. Second anchor may be a cylindrical anchor that is inserted into a prepared hole within a tissue. It is preferable that the entire length of the suture 470 includes a braided suture with a braided core (25a, 25b), except for the locking length(s) 20. Construct 470 may include a single locking length 20 or a plurality thereof spaced apart along the construct 470. Further examples of adjustable suture constructs for graft or tissue suspension or stabilization are also disclosed in commonly assigned provisional application 62/862,807 filed on Jun. 18, 2019 and titled “Methods and Devices for Tissue Graft Fixation”; herein incorporated in its entirety by reference.

FIG. 4C is a representation of a cross section through locking length 420a of FIG. 4A. The strands 5 are not shown to scale, for simplicity. The strands 5 are all preferably the same size between the outer rim 22 and the braided core portion extending therethrough.

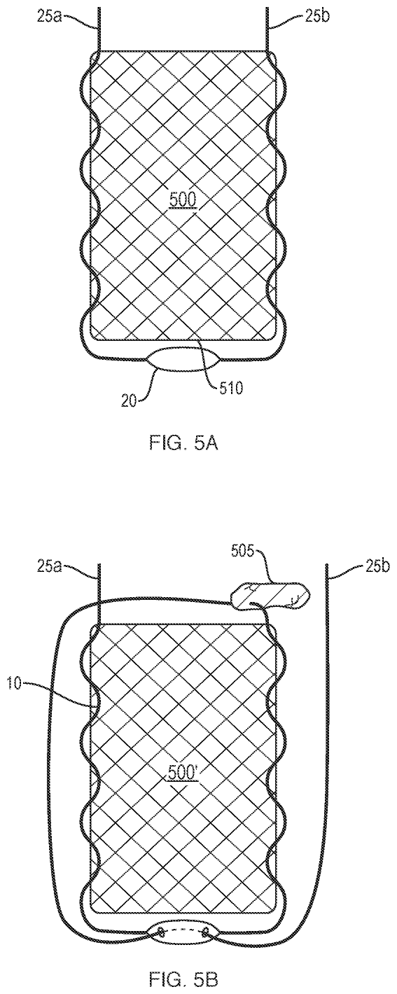

FIG. 5A and 5B illustrates an exemplary soft anchor 500 operatively coupled to braided suture construct 10. Soft anchor 500 is represented as a tubular body formed of suture braids and may be similar to an all-suture anchor disclosed in commonly owned U.S. Pat. No. 9,962,149 herein incorporated by reference in its entirety. Soft anchors however come in many forms known in the art; generally including predominantly soft, flexible and/or braided material having a first configuration for inserting into biological tissue such as a bone tunnel. Soft anchors also include a second or deployed configuration, that is oftentimes deployed by placing tension on a flexible strand or suture that extends along and is operative coupled to a length of the soft anchor. This suture or flexible strand may be defined as the “deploying suture”. A deployed soft anchor has a different shape from the first configuration in that it is both laterally deformed and longitudinally deformed. It is generally laterally expanded and longitudinally compressed compared to the first configuration. Soft anchors may come in other forms than a longitudinal tubular shape as shown, including flat tape that deforms into a series of S-beds, tubes in a U-bend shape, or tubes in a W-bend shape for example. Soft anchors are not precluded from including at least a portion including material that is rigid.

Suture 10 is shown operatively coupled to the soft suture anchor, and may be interwoven repeatedly through a portion of the anchor 500. Suture 10 may be the “deploying suture”. Alternatively, suture 10 may be operatively coupled to the anchor 500 and a repair tissue 505 and therefore be defined as the “repair suture”. Suture may be threaded through a portion of the anchor 500 to form an adjustable loop similar to loop construct 100. Suture 10 may thread through a portion of the anchor 500 and form a single adjustable loop only, as illustrated in FIG. 5B. In some embodiments, suture construct 10 may act as both the deploying suture and repair suture. As such, this disclosure includes a suture such as a suture 10, operatively coupled to a soft anchor 500 coupling the repair tissue 505 with the anchor 500.

Soft anchors 500 that are knotlessly locked have been described in at least commonly owned U.S. Pat. No. 9,962,150, herein incorporated by reference in its entirety. Soft anchor 500 may be operatively coupled to suture 10 as shown in FIG. 5A, with locking length 20 adjacent anchor 500. Locking length 20 may be at a distal end 510 of anchor as shown, or may be along a length of the anchor 500 or across a top or proximal end of anchor 500. Locking length 20 may be proximally spaced from anchor 500. Locking length 20 may have a length approximately equal to or greater than lateral width or diameter of soft anchor 500. Suture 10 may form two adjustable loops similar to that described in FIG. 2B, with at least a portion threaded between braids of the anchor 500 to operatively couple the suture 10 with anchor 500. Tension on at least one limb 25a, 25b may deploy anchor 500, which may laterally and longitudinally deform anchor 500, represented in FIG. 5B. Limbs 25a and 25b may be coupled to tissue 505 and a first tension on at least one limb 25a or 25b may draw the tissue 505 towards the anchor 500. Further tension may knotlessly lock the tissue 505 with the anchor 500 and may lock the anchor 500 in the second, deployed configuration. Alternative configurations may include a plurality of locking lengths 20. For example, a first locking length 20 may extend along a first length of the anchor 500 on a first side, and a second locking length may extend along the anchor 500 on a second different side of the anchor 500, or proximally spaced from anchor 500.

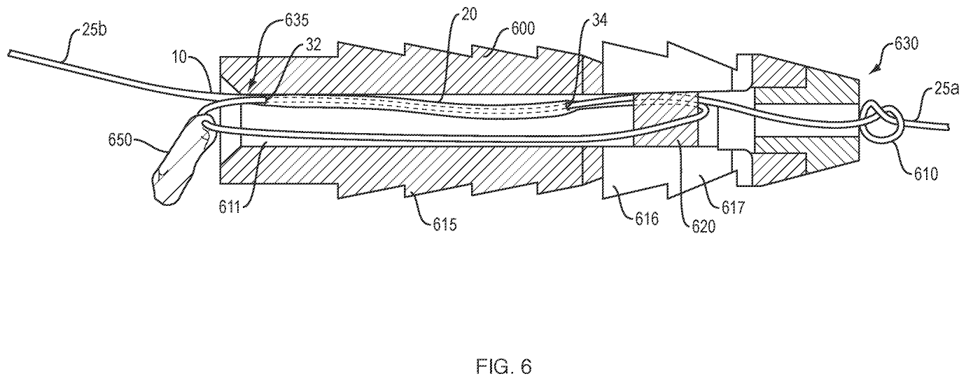

FIG. 6 shows an example of a knotless suture construct operatively coupled to a rigid anchor 600. Rigid anchor 600 may be sized to form an interference fit within a tissue such as a bone hole, or may have a deployable portion to laterally expand and lock with the target tissue. For example, a plug may be axially slid into an opening of the rigid anchor to laterally deform an outer portion of the anchor, which may include wings or barbs. Rigid anchor 600 is operatively coupled to a suture loop construct formed of suture 10 and may include a single loop (shown in FIG. 6) or a double loop construct shown in FIGS. 2a, 2B. Suture 10 may be operatively coupled using a knot 610 to the anchor. In the particular exemplary embodiment illustrated in FIG. 6, rigid anchor 600 may include a plurality of barbs 615 extending circumferentially around it. Alternative embodiments may include external threads or flexible wings, and anchor 600 may be a screw-in anchor or a push-in style anchor. Transverse openings/channels 616 and 617 allow suture 10 and/or suture passing device(s) to pass around bar 620. Cannulation 611 extends along the anchor 600 to allow passage of suture 10 and or passing devices. In alternative embodiments suture 10 may have an alternative routing around anchor 600, eliminating the bar 620. For example suture 10 may extend along a length of cannulation 611 and around a distal-most end 630 of anchor and along an outer wall of anchor (not shown) back to a proximal end 635. Outer wall may include elongate slots to receive the length of the suture therealong and provide space between a bone tunnel wall and the suture 10. An example embodiment may include anchors with elongate slots as shown in commonly owned U.S. Pat. No. 8,758,367 incorporated by reference in its entirety.

Returning to the example embodiment in FIG. 6, suture 10, as previously described may be preloaded within or along anchor 600 and may be operatively coupled by tying static knot 610. Alternatively suture 10 may be coupled using insert molding, adhesive, welding or by any other means known in the art. Suture 10 is shown directly coupled at a distal-most end of anchor 600, but may alternatively be directly coupled along a length of the suture 10 that lies along a portion of the anchor 600, proximally disposed therefrom. As shown, suture 10 may pass round bar 620, which is large enough to allow suture 10 to take gradual turns instead of sharp turns. Suture 10 then passes through anchor cannulation 611.

Anchor may be provided with a suture passer such as a snare or wire (not shown) threaded through locking length 20 to provide a means of easily threading a limb (25b) though the locking length 20. Suture passer may include a loop to snare suture limb 25b and may enter and exit locking length 20 at apertures 32 and 34, similar to those described in FIG. 2A. Locking length 20 may be approximately shorter than anchor length from distal most 630 end to proximal most end 635. Preferably, suture loop construct may include a braided core segment (limb 25a and 25b) that forms a substantial portion of the suture construct. Preferably, a ratio of the length with a braided core to a locking length is greater than 1:1 to provide reduced elongation due to physiological cyclic loading.

Once anchor 600 is inserted into bone, using an inserter device (not shown), suture limb 25b may be coupled to tissue 650 and then drawn through locking length 20 using suture passer (not shown). A first tension may then draw tissue 650 towards the anchor 600 proximal end 635 and therefore the bone. Further tension may activate the locking length 20 so as to knotlessly lock the suture loop and thereby the tissue 650 with anchor 600.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A tissue repair system comprising:
a suture construct including a first and second braided core segment and a locking length therebetween, wherein the locking length is continuously braided with the first and second braided core segment and defines a longitudinal passage therethrough;
wherein the suture construct includes at least one adjustable loop with the first braided core segment disposed along the longitudinal passage;
wherein tension on at least one of the first or second braided core segments is configured to knotlessly lock the locking length around the first braided core segment disposed therein.

2. The tissue repair system of claim 1 wherein the suture construct includes at least two adjustable loops with the first and second braided core segment disposed along the longitudinal passage; and wherein tension on at least one of the first or second braided core segments knotlessly locks the locking length around the first and second braided core segment disposed therein

3. The tissue repair system of claim 1, wherein the suture construct is operatively coupled to an anchor.

4. The tissue repair system of claim 3, wherein the at least one adjustable loop is configured to suspend a ligament in a bone hole.

5. The tissue repair system of claim 2 wherein the locking length defines a cradle for operatively coupling with a ligament.

6. The tissue repair system of claim 1 wherein the locking length has a first length and the first and second braided core segments define a second length, the second length more than twice as long as the first length.

7. The tissue repair system of claim 1 wherein the locking length and first and second braided core segment have the same number of strands.

8. The tissue repair system of claim 1 wherein all of the strands in the first and second braided core segment are disposed along the locking length.

9. The tissue repair system of claim 2 wherein the first and second braided core segments pass through the locking length in opposite directions.

10. A knotless tissue fixation system comprising:
a tissue anchor; and
a continuously braided suture operatively coupled to the tissue anchor, the braided suture having a braided core therealong, interrupted by a coreless portion, defining a locking length;
wherein the locking length defines a predetermined limited length sufficient to knotlessly lock around a length of suture disposed within the locking length upon tension being applied to the continuously braided suture.

11. The knotless tissue fixation system of claim 10 wherein the length of suture disposed along the locking length is the continuously braided suture including the braided core.

12. The knotless tissue fixation system of claim 10 wherein the length of suture disposed along the locking length portion is a separate suture, independently formed from the braided suture.

13. The knotless tissue fixation system of claim 10 wherein the tissue anchor comprises at least one tissue anchor selected from the group consisting of a soft anchor, a rigid anchor and a cortical button.

14. The knotless tissue fixation system of claim 10 wherein the continuously braided suture has the same number of strands throughout.

15. The knotless tissue fixation system of claim 10 wherein the locking length has a length that is limited to a minimum length sufficient to securely knotlessly lock around the suture.

16. The knotless tissue fixation system of claim 10 wherein the locking length has a first length sufficient to lock around the length of suture disposed therein, and the entire continuously braided suture defines a second length, the second length more than twice the first length.

17. A method of coupling a first tissue to a second tissue comprising:
coupling a suture to the first tissue, wherein the suture includes a locking length extending between a first and second braided core segments of the suture; wherein both braided core segments and the locking length are continuously braided with each other and have equal number of strands to each other therealong;
placing a braided core segment into and along the locking length;
applying a first tension to a first limb of the suture to draw the first tissue towards a target location; and
applying a second tension to the suture, to knotlessly lock the locking length around the braided core segment disposed therein and thereby knotlessly fix the tissue or graft.

18. The method of claim 18 further comprising engaging an anchor with a second tissue, the anchor operatively coupled to the suture.

19. The method of claim 17 further comprising placing another braided core segment of the suture into and along the locking length before applying a second tension.

20. The method of claim 18 further comprising placing another braided core segment of the suture into and along another locking length of the suture before applying a second tension.
